(19) United States
(12) Patent Application Publication
(10) Pub. No.: US 2009/0118514 A1
(43) Pub. Date: May 7, 2009

(54) PROCESSES FOR PREPARING PIOGLITAZONE AND ITS PHARMACEUTICALLY ACCEPTABLE SALTS

(76) Inventors: Raghupathi Reddy Anumula, Hyderabad (IN); Goverdhan Gilla, Hyderabad (IN); Lokeswara Rao Madivada, Krishna District (IN); Prabhakar Macharla, Hyderabad (IN); Kavitha Charagondla, Nalgonda (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/265,142

(22) Filed: Nov. 5, 2008

Related U.S. Application Data
(60) Provisional application No. 61/079,925, filed on Jul. 11, 2008.

(30) Foreign Application Priority Data
Nov. 6, 2007 (IN) 2555/CHE/2007

(51) Int. Cl. C07D 417/12

(52) U.S. Cl. 546/269.7

(57) ABSTRACT

An improved process for the preparation of pioglitazone and its pharmaceutically acceptable salts by reducing 5-[4-[2-(5-ethyl-2-pyridyl)ethoxy]benzilidene]-2,4-thiazolidinedione with sodium borohydride in presence of a cobalt ion and dimethyl glyoxime. More particularly the cobalt ion used is cobalt chloride hexahydrate, or, cobalt (II) nitrate hexahydrate in presence of mixture of DMF and water as solvents.

### INTRODUCTION

The present invention relates to pioglitazone and its pharmaceutically acceptable salts, processes for preparing pioglitazone and its pharmaceutically acceptable salts, and pharmaceutical compositions thereof.

Pioglitazone hydrochloride is the adopted name for a compound having the chemical name 5-[4-2-(5-ethyl-2-pyridyl)ethoxy]-benzyl]-2,4-thiazolidinedione hydrochloride and is represented by the structural Formula I.

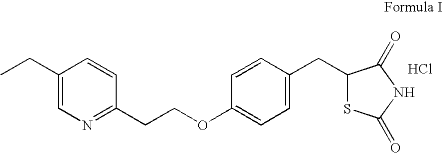

Pioglitazone is useful to manage type 2 diabetes and is marketed as hydrochloride salt under the brand name ACTOS®.

Meguro et al., in U.S. Pat. No. 4,687,777, disclose a process for the preparation of pioglitazone hydrochloride and its homologues, which process involves reacting 2-(5-ethyl-2-pyridyl)ethanol with 4-fluoronitro benzene in the presence of sodium hydride in dimethyl formamide to give 4-{2-(5-ethyl-2-pyridyl)ethoxy] nitrobenzene, further reducing with palladium carbon and resulting amine on diazotisation, and reacting with methyl acrylate and thiourea to afford 5-[4-[2-(5-ethyl-2-pyridyl)ethoxy]benzyl]-2-imino-4-thazolidinedione. The imino thiazolidinedione was subjected to hydrolysis in presence of HCl to give pioglitazone hydrochloride.

Meguro et al., in U.S. Pat. No. 4,812,570, disclose a process for the preparation of pioglitazone in which 5-ethyl-2-pyridine ethanol is reacted with p-toluenesulfonyl chloride in presence of sodium hydroxide as base and methylene chloride as solvent, which on further reaction with p-hydroxy benzaldehyde in presence sodium hydroxide gave 4-[2-(5-ethyl-2-pyridyl)ethoxy]benzaldehyde.

The obtained benzaldehyde derivative was further reacted with thiazolidinedione in presence of piperidine and ethanol as solvent to afford 5-{4-[2-(6-methyl-2-pyridyl)ethoxy]benziyliden}-2,4-thiazolidinedione, which, on reduction using palladium on carbon in dioxane solvent, gave pioglitazone free base.

Huber et al., U.S. Pat. No. 5,585,495, discloses reduction of 5-[4-2-(5-ethyl-2-pyridyl)ethoxy]-benzyliden]-2,4-thiazolidinedione using sodium borohydride in presence of cobalt ion, a ligand, and aqueous THF as solvent to afford pioglitazone free base

The above-mentioned processes suffer serious disadvantages due to the formation of impurities because of over reduction.

Thus, there is a need for simple, cost effective, efficient, and industrially feasible processes for the synthesis of pioglitazone hydrochloride of Formula I.

### SUMMARY

There are provided processes for the preparation of a substantially pure pioglitazone of Formula I or its pharmaceutically acceptable salts, which processes are simple, cost effective, and easy to operate on commercial scale.

The present invention includes processes for the preparation of pioglitazone or a pharmaceutically acceptable salt thereof, which processes comprise at one of the steps of:
(1) reacting the compound 5-ethyl-2-methyl pyridine of Formula IX with formaldehyde at suitable temperature to afford 2-(5-ethyl-2-pyridyl)ethanol of Formula VIII

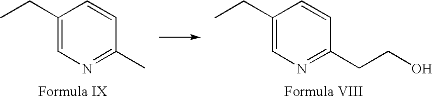

(2) reacting 2-(5-ethyl-2-pyridyl)ethanol of Formula VIII obtained in step (1) with methane sulfonyl chloride in presence of suitable base and suitable solvent to afford (5-ethyl-2-pyridyl)-ethyl methanesulfonate of Formula VIII;

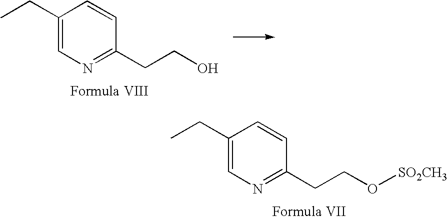

(3) reacting the compound of Formula VII with 4-hydroxybenzaldehyde of Formula VI in presence of suitable base and solvent to afford 4-[2-(5-ethyl-pyridin-2-yl)-ethoxy]-benzaldehyde of Formula V;

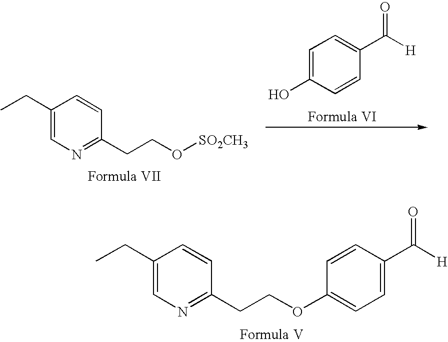

(4) reacting the compound of Formula V with 2,4-thiazolidinedione of Formula IV to afford 5-[4-[2-(5-ethyl-2-pyridyl)ethoxy]benzylidene]-2,4-thiazolidinedione of Formula III;

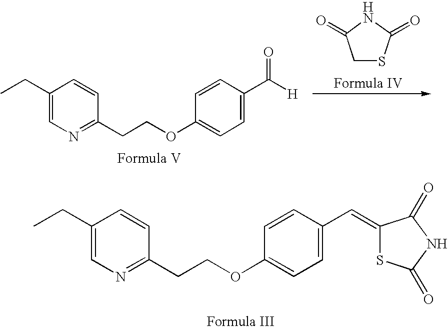

(5) reducing 5-[4-[2-(5-ethyl-2-pyridyl)ethoxy]benzylidene]-2,4-thiazolidinedione of Formula III with sodium borohydride in presence of suitable cobalt ion, and ligand and suitable organic solvent to afford pioglitazone free base, namely 5-[4-2-(5-ethyl-2-pyridyl)ethoxy]-benzyl]-2,4-thiazolidinedione of Formula II; and

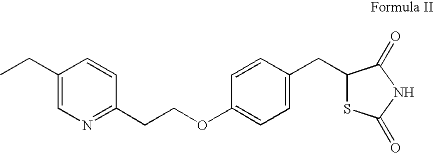

(6) reacting pioglitazone free base with hydrochloric acid in presence of suitable solvent to afford pioglitazone hydrochloride, namely 5-[4-2-(5-ethyl-2-pyridyl)ethoxy]-benzyl]-2,4-thiazolidinedione hydrochloride of Formula I.

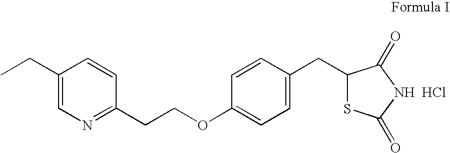

The present invention includes pharmaceutical compositions comprising a therapeutically effective amount of pioglitazone or its pharmaceutically acceptable salts and one or more pharmaceutically acceptable carriers, excipients, or diluents.

### BRIEF DESCRIPTION OF THE DRAWINGS

FIG. 1 is a schematic representation of a process for the preparation of the compound of Formula I.

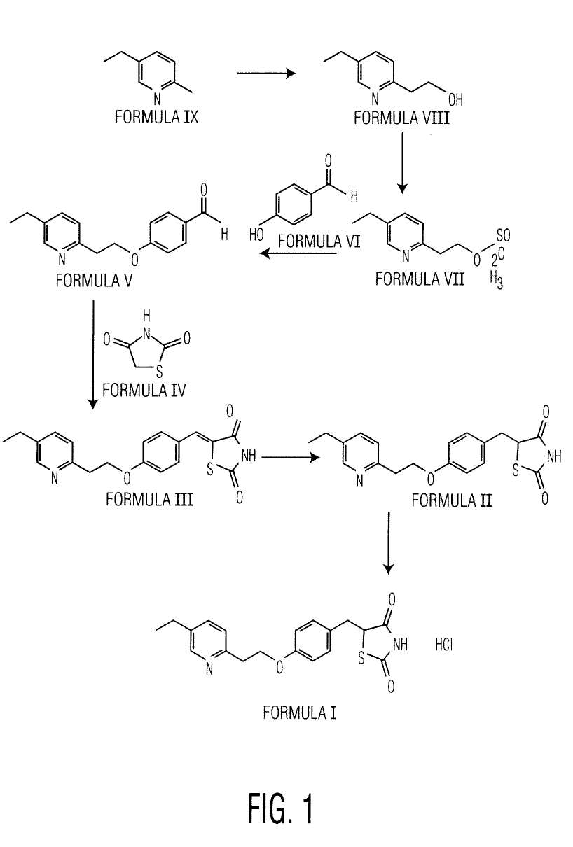

FIG. 2 is an X-ray powder diffraction (XRPD) pattern of Pioglitazone freebase.

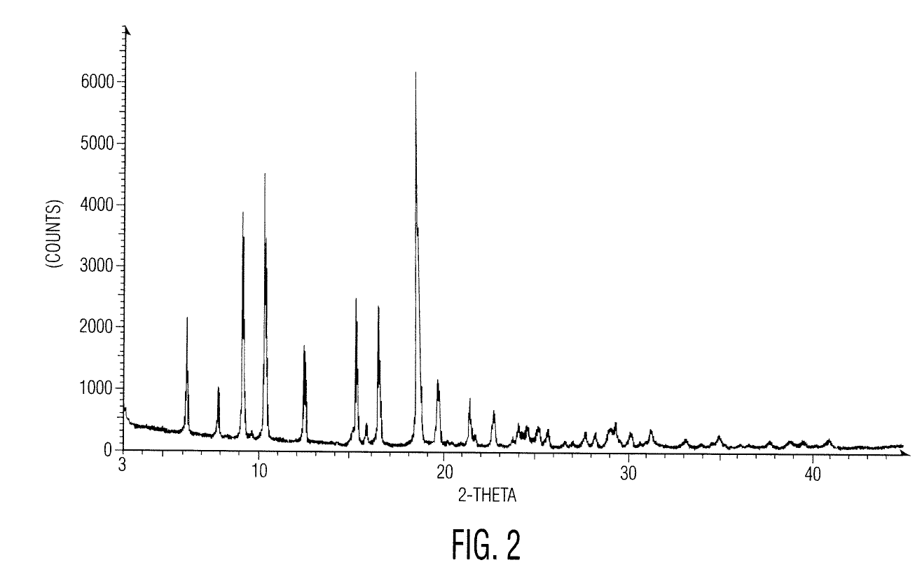

FIG. 3 is an DSC pattern of Pioglitazone freebase.

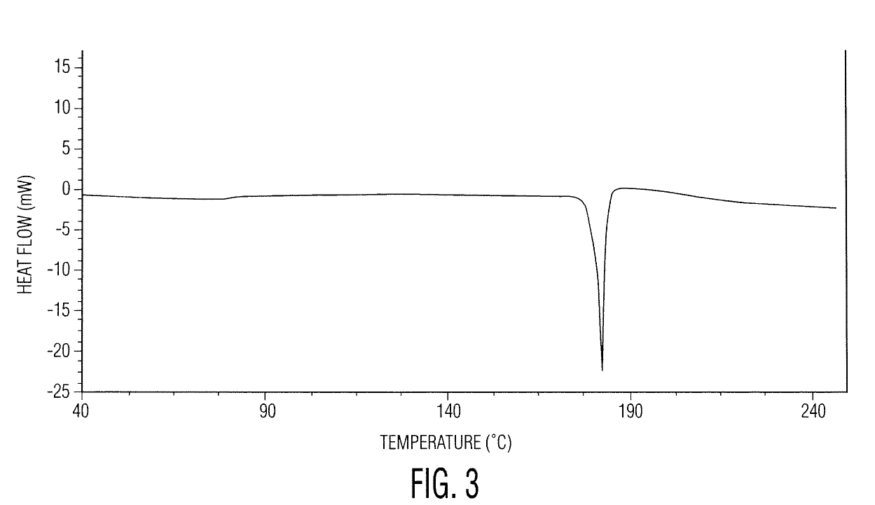

FIG. 4 is an X-ray powder diffraction (XRPD) pattern of Pioglitazone hydrochloride.

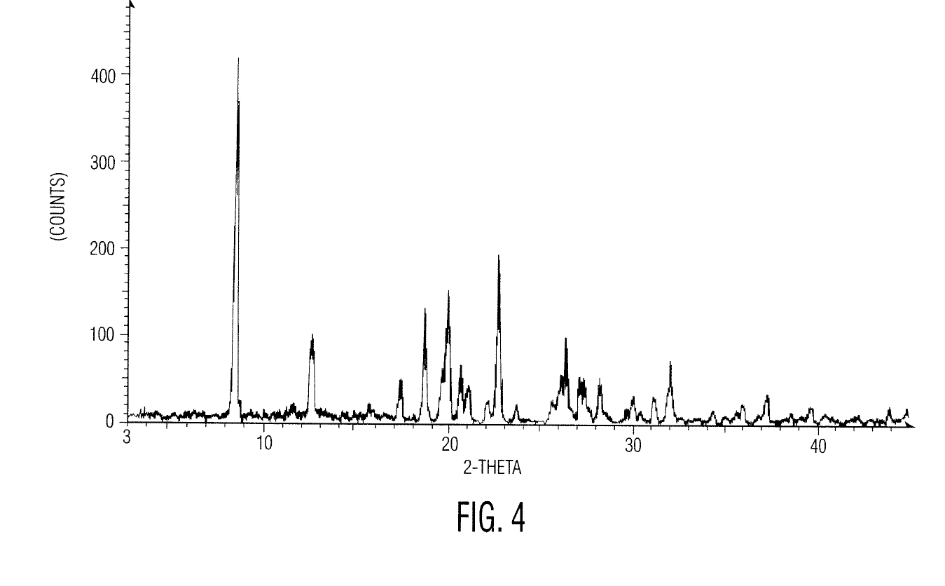

FIG. 5 is an (DSC) pattern of Pioglitazone Hydrochloride.

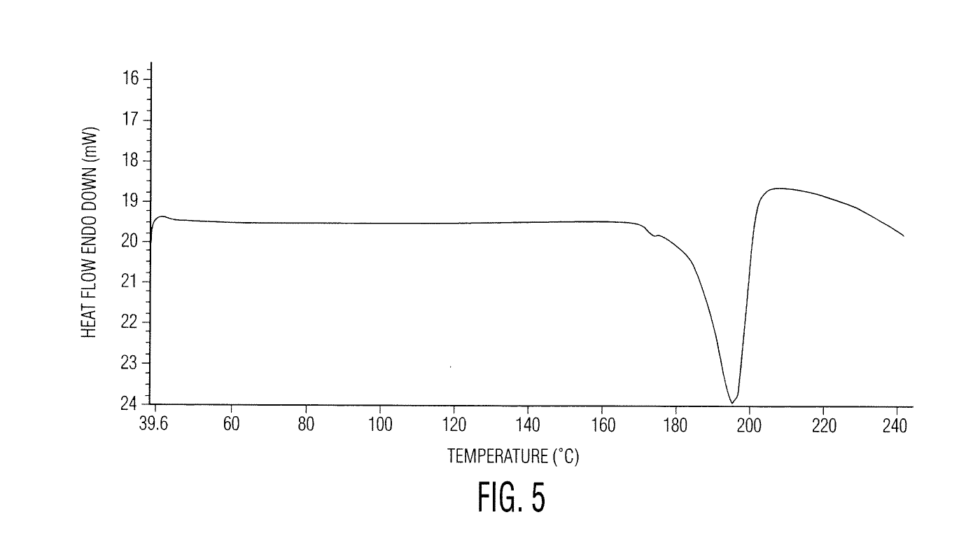

FIG. 6 is an (IR) spectrum of pioglitazone freebase.

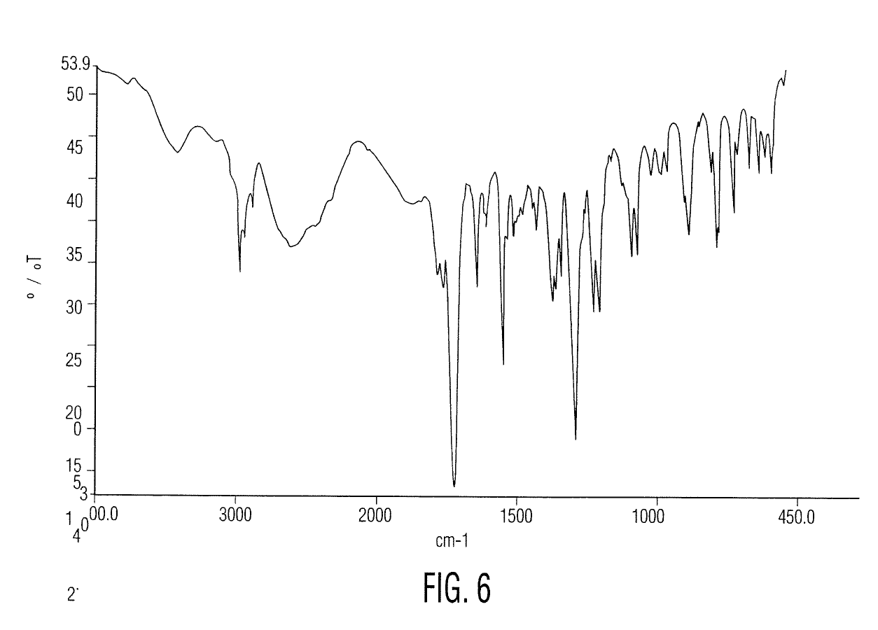

FIG. 7 is an (IR) spectrum of pioglitazone hydrochloride.

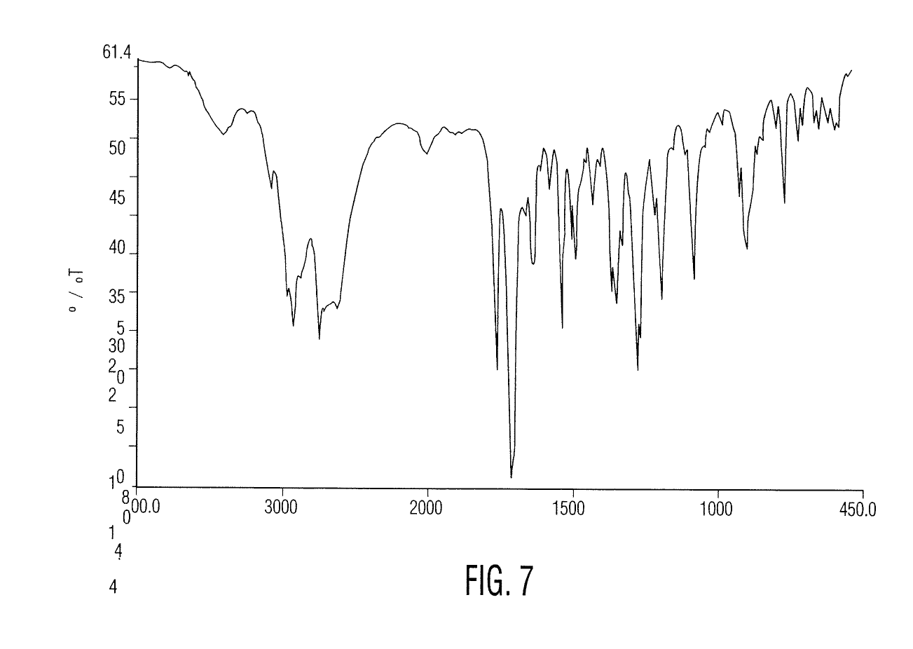

### DETAILED DESCRIPTION

Pioglitazone and its salts may be described by reference to patterns, spectra, or other graphical data as “substantially” shown or depicted in a figure, or by one or more data points. It will be appreciated that patterns, spectra, and other graphical data can be shifted in their positions, relative intensities, or other values due to a number of factors known to those of skill in the art. For example, in the crystallographic and powder X-ray diffraction arts, shifts in peak positions or the relative intensities of one or more peaks of a pattern can occur because of, without limitation: the equipment used the sample preparation protocol, preferred packing and orientations, the radiation source, operator error, method and length of data collection, and the like. However, those of ordinary skill in the art should be able to compare the figures herein with a pattern generated of an unknown form of, in this case, lopinavir, and confirm its identity as one of the forms disclosed and claimed herein. The same holds true for other techniques which may be reported herein.

In addition, where a reference is made to a figure, it is permissible to, and this document includes and contemplates, the selection of any number of data points illustrated in the figure that uniquely define that crystalline form, salt, solvate, and/or optical isomer, within any associated and recited margin of error, for purposes of identification.

All percentages and ratios used herein are by weight of the total composition and all measurements made are at about 25° C. and about normal pressure unless otherwise designated. All temperatures are in degrees Celsius unless specified otherwise. As used herein, “comprising” (open ended) means the elements recited, or their equivalent in structure or function, plus any other element or elements which are not recited. The terms “having” and “including” are also to be construed as open ended. As used herein, “consisting essentially of” means that the invention may include ingredients in addition to those recited in the claim, but only if the additional ingredients do not materially alter the basic and novel characteristics of the claimed invention. All ranges recited herein include the endpoints, including those that recite a range “between” two values. Whether so indicated or not, all values recited herein are approximate as defined by the circumstances, including the degree of expected experimental error, technique error, and instrument error for a given technique used to measure a value.

The present invention includes processes for the preparation of pioglitazone or a pharmaceutically acceptable salt thereof, which processes comprise at least one of the steps of:
(1) reacting the compound 5-ethyl-2-methyl pyridine of Formula IX with formaldehyde at suitable temperature and at suitable reaction conditions to afford 2-(5-ethyl-2-pyridyl)ethanol of formula VIII;
(2) reacting the 2-(5-ethyl-2-pyridyl)ethanol of Formula VIII with methane sulfonyl chloride in presence of a suitable base and suitable solvent to afford (5-ethyl-2-pyridyl)-ethyl methanesulfonate of Formula VIII;
(3) reacting the (5-ethyl-2-pyridyl)-ethyl methanesulfonate of Formula VII with 4-hydroxybenzaldehyde of Formula VI in presence of a suitable base and solvent to afford 4-[2-(5-ethyl-pyridin-2-yl)-ethoxy]-benzaldehyde of Formula V;
(4) reacting the 4-[2-(5-ethyl-pyridin-2-yl)-ethoxy]-benzaldehyde of Formula V with 2,4-thiozolidinedione of Formula IV to afford 5-[4-[2-(5-ethyl-2-pyridyl)ethoxy]benzilidene]-2,4-thiazolidinedione of Formula III;
(5) reducing the 5-[4-[2-(5-ethyl-2-pyridyl)ethoxy]benzilidene]-2,4-thiazolidinedione of Formula III with sodium borohydride in presence of a suitable cobalt ion, dimethyl glyoxime as ligand, and a suitable organic solvent to afford pioglitazone free base, namely 5-[4-2-(5-ethyl-2-pyridyl)ethoxy]-benzyl]-2,4-thiazolidinedione of Formula II; and
(6) reacting pioglitazone free base with hydrochloric acid in the presence of a suitable solvent to afford pioglitazone hydrochloride, namely 5-[4-2-(5-ethyl-2-pyridyl)ethoxy]-benzyl]-2,4-thiazolidinedione hydrochloride of Formula I.

In step (1), the compound of Formula IX is reacted with aqueous formaldehyde at suitable temperatures. Suitable temperatures for conducting the reaction can range from about 50° C. to about 170° C., and suitable time can range from about 1 hour to about 15 hours or about 1 hour to about 3 hours. The obtained product may be isolated by using high vacuum distillation. Suitable temperature for distillation can range from about 100° C. to about 130° C. and suitable vacuum for distillation can range from to about 500 to about 800 mm Hg to afford 2-(5-ethyl-2-pyridyl)ethanol.

Organic bases that may be use in step (2) include and are not limited to C₁-C₁₀, straight chain, branched or cyclic primary, secondary, or tertiary, aromatic or aliphatic amines, such as, for example, tert butyl amine, dicyclohexyl amine, dimethylamine, triethylamine, diisopropyl amine, and diisopropyl ethylamine. For example, the organic base may be triethyl amine. The molar ratio of the organic base to the compound of Formula III can range from about 1 to 5, or about 1 to 2.

The reaction may be carried out in presence or absence of a solvent. Suitable solvents include and are not limited to: halogenated hydrocarbons, such as, for example, dichloromethane, ethylene dichloride, and chloroform; hydrocarbons, such as, for example, benzene, toluene, hexane, and cyclohexane; aprotic solvents, such as, for example, N,N-dimethylformamide (DMF), dimethylsulfoxide (DMSO), and N,N-dimethylacetamide (DMA); and mixtures thereof in various proportions.

Suitable temperatures for conducting the reaction of step (2) can range from about 10° C. to about 50° C., and the suitable time can range from about 1 hour to about 10 hours or about 2 hours to about 3 hours. The product may be isolated by usual work-up procedure to afford the compound of Formula VIII.

In step (3), the obtained compound of Formula VIII is reacted with Formula VI in the presence of a base, and under suitable reaction conditions. Suitable bases include and are not limited to inorganic bases, such as, for example, sodium hydroxide, potassium hydroxide, sodium carbonate, sodium bicarbonate, potassium bicarbonate, potassium carbonate, sodium methoxide, and potassium methoxide. The base may optionally be used in combination with water.

The molar ratio of a base to a compound of Formula II can range from about 1 to 5. The product may be isolated by usual work-up procedure to afford the compound of Formula V.

In step (4), the obtained compound of Formula V is further reacted with 2,4-thiozolidinedione of Formula VI in presence of piperidine and methanol as solvent at suitable reaction conditions. Suitable temperatures for conducting the reaction can range from about 10° C. to about 80° C., and the suitable time can range from about 5 hours to about 20 hours or about 12 hours to about 14 hours. The product may be extracted by usual work-up procedure to afford the compound of Formula VIII.

In step (5), the compound of Formula III is reduced into compound of Formula II by using sodium borohydride in the presence of cobalt ion and dimethyl glyoxime as ligand and suitable organic solvent at suitable reaction conditions. For example, the cobalt ion is in the form of cobaltous chloride, cobaltous nitrate, cobaltous sulphate, cobaltous ammonium sulphate, cobaltous oxalate, cobaltous hydroxide, and/or cobaltous carbonate.

Suitable solvents that can be used in this step (5) include and are not limited to: alcohols, such as, for example, methanol, ethanol, isopropyl alcohol, and n-butanol; ketones, such as, for example, acetone, ethyl methyl ketone, and methyl isobutyl ketone; esters, such as, for example, ethyl acetate, n-propyl acetate, n-butyl acetate, and t-butyl acetate; ethers, such as, for example, THF, 1,4-dioxane; aprotic solvents, such as, for example, N,N-dimethylformamide (DMF), dimethylsulfoxide (DMSO), and N,N-dimethylacetamide (DMA); and protic solvents, such as, for example, water and the aforementioned alcohols. When used, the protic solvent may be present in the aprotic solvent in an amount about 1% to about 5% by volume. The obtained compound can be optionally treated with or without charcoal in presence of solvents.

Optionally, the obtained compound can be purified by recrystallization. Suitable solvents that can be used for recrystallization can be dimethyl formamide, acetone, methanol, water, 1,4 dioxane, isopropyl alcohol, or mixtures there of with various proportions.

In step (6), the conversion of pioglitazone freebase to pharmaceutically acceptable salt may be achieved by treating the pioglitazone base with an acid. The phrase “pharmaceutically acceptable salts of pioglitazone” as used herein refer to salts prepared from pharmaceutically acceptable non-toxic acids, including inorganic acids, such as, for example, hydrochloric acid and hydrobromic acid, and organic acids, such as, for example, acetic acid, tartaric acid, and methanesulfonic acid. For example, the conversion of pioglitazone freebase to hydrochloride salt may be achieved by treating the solution of pioglitazone base with a source of hydrochloric acid. Sources of hydrochloric acid that can be used include and are not limited to aqueous hydrochloric acid, hydrogen chloride gas purged in suitable organic solvent, and ammonium chloride (NH₄Cl).

Suitable solvents used to for salt formation include and are not limited to alcoholic solvents, such as, for example, methanol, ethanol, and isopropanol, ketonic solvents, such as, for example, acetone, methyl isobutyl ketone, and the like, and water, and the mixtures thereof with various proportions.

The present invention includes 5-[4-[2-(5-ethyl-2-pyridyl)ethoxy]benzylidene]-2,4-thiazolidinedione of Formula (III) substantially free from the process-related impurity 5-{4-[2-(5-Ethyl-pyridin-2-yl)ethoxy]-benzylidene}-3-[2-(5-ethyl-pyridin-2-yl)-ethyl]-thiazolidine-2,4-dione of Formula (XI) at relative retention time of about 1.4 RRT by HPLC.

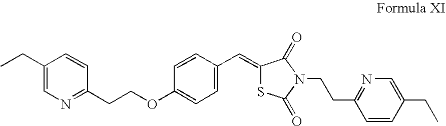

The present invention includes pioglitazone free base of Formula (II) substantially free from the potential process-related impurity 5-{4-[2-(5-Ethyl-pyridin-2-yl)-ethoxy]-benzyl}-3-[2-(5-ethyl-pyridin-2-yl)-ethyl]-thiazolidine-dione Formula (XII).

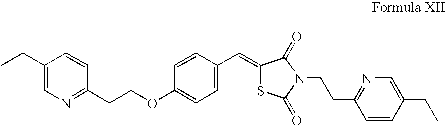

The above-mentioned impurities are analyzed by high performance liquid chromatography (HPLC) method using a symmetry shield RP-18, 250×4.6 mm×5 μm columns with the following parameters.

| Flow Rate | 0.8 mL/min | | | | | | |
|---|---|---|---|---|---|---|---|
| Detector | UV detector and integrator | | | | | | |
| Injection Load | 40 μL | | | | | | |
| Temperature | ambient | | | | | | |
| Run Time | 70 minutes | | | | | | |
| Mobile Phase | degassed buffer | | | | | | |
| A | | | | | | | |
| Buffer | dissolve 1.36 g of potassium dihydrogen phosphate, | | | | | | |
| preparation | 0.87 g of dipotassium hydrogen phosphate of water | | | | | | |
| | and 1 g of 1-decane sulphonic acid in 1000 ml of | | | | | | |
| | water and adjust the pH to 3 with dilute phospho- | | | | | | |
| | ric acid. | | | | | | |
| Mobile phase | acetonirile, methanol, and water in the ratio of | | | | | | |
| B | 700:200:100 (v/v) | | | | | | |
| Gradient | Time (min) | 0.01 | 10 | 35 | 50 | 55 | 70 |
| Progamme: | % A | 60 | 60 | 30 | 30 | 60 | 60 |
| | % B | 40 | 40 | 70 | 70 | 40 | 40 |

The present invention includes a particle size distribution of pioglitazone hydrochloride having a mean particle size of less than or equal to 200 μm before micronization and having mean particle size of lesser than or equal to 100 μm after micronisation.

The D₁₀, and D₉₀ values are useful ways for indicating a particle size distribution. D₉₀ refers to at least 90 volume percent of the particles having a size smaller than the said value. Likewise, D₁₀ refers to 10 volume percent of the particles having a size smaller than the said value. D₅₀ refers to at least 50 volume percent of the particles having a size smaller than the said value. Methods for determining D₁₀, D₅₀ and D₉₀ include laser diffraction.

Pioglitazone hydrochloride according to the present invention may have particles before micronisation having a D₁₀ of less than about 10 μm or less than about 4 μm; D₅₀ less than about 75 μm or less than about 50 μm or less than about 20 μm; and D₉₀ less than about 300 μm or less than about 150 μm. There is no specific lower limit for any of the D values.

Pioglitazone hydrochloride according to the present invention may have particles after micronisation having a D₁₀ of less than about 5 μm or less than about 2 μm; D₅₀ less than about 30 μm or less than about 25 μm or less than about 19 μm; and D₉₀ of less than about 100 μm or less than about 50 μm. There is no specific lower limit for any of the D values.

Certain specific aspects and embodiments of the present application are further described in the examples given below that are provided by the way of illustration only and, therefore, should not be construed to limit the scope of the present application.

### EXAMPLES

### Example 1

### Preparation of 2-(5-Ethyl-2-Pyridyl)Ethanol (Formula VIII)

5-Ethyl-2-methyl pyridine (200 g) of Formula (IX) and 37% aqueous formaldehyde solution (134 g) are charged into an autoclave and heated to about 150° C. to about 160° C. The resultant reaction mass is maintained at that temperature for about 3 hours to complete the reaction. The reaction mixture is then cooled to about 25° C. The obtained reaction mass is distilled for about 500 to about 800 mm Hg at about 80° C. to afford 58 g of the title compound.

### Example 2

### Preparation of 5-[4-[5-Ethyl-2-Pyridyl)Ethoxy]Benzilidene]-2,4-Thiazolidinedione (Formula III)

2-(5-Ethyl-2-pyridyl)ethanol (50 g) and toluene (200 mL) are charged into a round bottom flask and stirred for about 10 minutes at about 25° C. followed by addition of triethyl amine (57.6 mL). Methane sulfonyl chloride (42.5 g) is added slowly through a dropper for about 25 minutes at about 25° C. and stirred for about 3 hours at about 25° C. The separated solid is filtered and washed with toluene (60 mL). The obtained filtrate is washed with 4% sodium bicarbonate solution. The layers are separated and aqueous layer is extracted with toluene (50 mL). The organic layers are combined and washed with water (2×60 mL). The resultant organic layer is charged into a round bottom flask followed by addition of 4-hydroxy benzaldehyde (43.2 g) and potassium carbonate (80 g) and heated to about 90° C. for about 14 hours and then cooled to about 50° C. The obtained reaction mass is quenched by addition of water (250 mL) and stirred for about 30 minutes. The layers are separated and the aqueous layer is extracted with toluene (2×150 ml). The organic layers are combined and washed with 5% sodium hydroxide solution. The separated aqueous layer is extracted by toluene (2×32 mL). The separated organic layers are combined and washed with water (2×125 mL). The resultant clear organic layer is distilled off completely under vacuum below 65° C. to afford the crude compound of Formula (V).

The obtained crude of Formula V, methanol (450 mL) and piperidine (23.3 g) are charged in to a round bottom flask and heated to about 65° C. for about 14 hours. After completion of the reaction the reaction mass is cooled to about 25° C. and methanol (195 mL) is charged and it is stirred for about 15 minutes. The pH is adjusted to about 6 to about 6.5 by using acetic acid (35 mL) followed by addition of methanol (130 mL) and stirred for about 15 minutes. The obtained reaction solution is heated to about 65° C. and stirred for about 60 to about 90 minutes and then cooled to about 25° C. The separated solid is filtered and washed with methanol (65 mL) and suck dried. The obtained solid is further dried under vacuum at about 65° C. for about 4 hours to afford 50.7 g of the title compound.

HPLC purity: 97.1%

### Example 3

### Preparation of 5-[4-[5-Ethyl-2-Pyridyl)Ethoxy]Benzyl]-2,4-Thiazolidinedione (Formula II)

5-[4-[2-Ethyl-2-pyridyl)ethoxy]benzilidene]-2,4-thiazolinedione (20 g), methanol (40 mL), water (140 mL) and 4% sodium hydroxide solution (26 mL) are charged into a round bottom flask and stirred for about 10 minutes. Cobalt chloride hexahydrate (0.2 g) and dimethyl glyoxime are dissolved in dimethyl formamide (30 mL) and added slowly for about one hour at about 20° C. to about 35° C. Mixture of sodium borohydride (3.7 g dissolved in water 40 mL), and 4% sodium hydroxide solution (9 mL) is added slowly about 3 hours at about 20° C. and stirred for about 3 hours.

Charcoal (1 g) is added into the reaction mass and stirred for about 30 min at about 20° C. The reaction solution is passed through high flow bed. The resultant filtrate is charged into a fresh round bottom flask and the pH of the reaction solution is adjusted to about 6.5 to about 7.0 using acetic acid (8 mL) and it is stirred for solid separation at about 25° C. The separated solid is filtered and washed with water (20 mL). The obtained solid is further washed with methanol (2×25 mL) and suck dried. The obtained solid is further dried at about 70° C. for about 8 hours

The obtained solid is again charged into a round bottom flask containing dimethyl formamide (80 mL) and heated to about 90° C. to get clear solution. The resultant solution is cooled to about 25° C. and stirred for about 60 minutes for solid separation. The separated solid is filtered and washed with dimethyl formamide (20 mL). The obtained cake is further washed with acetone and suck dried. The obtained solid is further dried at about 70° C. for about 4 hours to afford 15.5 g of the title compound.

HPLC purity: 98.92%

### Example 4

### Preparation of 5-[4-[5-Ethyl-2-Pyridyl)Ethoxy]Benzyl]-2,4-Thaizolidinedione (Formula II) by Using Cobalt (II) Ammonium Sulfate

5-[4-[2-Ethyl-2-pyridyl)ethoxy]benzylidene]-2,4-thaizolidinedione (20 g), methanol (40 mL), water (140 mL) and 4% sodium hydroxide solution (26 mL) are charged into a round bottom flask and stirred for about 10 minutes. Cobalt (II) ammonium sulfate (0.12 g) and dimethyl glyoxime are dissolved in dimethyl formamide (30 mL) and added slowly for about one hour at about 20° C. to about 35° C. Mixture of sodium borohydride (3.7 g dissolved in water 40 mL), and 4% sodium hydroxide solution (9 mL) is added slowly about 3 hours at about 20° C. and stirred for about 3 hours.

Charcoal (1 g) is added into the reaction mass and stirred for about 30 minutes at about 20° C. The reaction solution is passed through high flow bed. The resultant filtrate is charged into the fresh round bottom flask and the pH of the reaction solution is adjusted to about 6.5 to about 7.0 using acetic acid (8.3 mL) and it is stirred for solid separation at about 25° C. The separated solid is filtered and washed with water (20 mL). The obtained solid is further washed with methanol (2×25 mL) and suck dried for about 10 to about 15 minutes. The obtained solid is further dried at about 70° C. for about 8 hours

The obtained solid is again charged into a round bottom flask containing dimethyl formamide (80 mL) and heated to about 90° C. to get clear solution. The resultant clear solution is cooled to about 25° C. and stirred for about 60 minutes for solid separation. The separated solid is filtered and washed with dimethyl formamide (20 mL). The obtained solid cake is further washed with acetone and suck dried. The obtained solid is further dried at about 70° C. for about 4 hours to afford 14.2 g of the title compound.

### Example 5

### Preparation of 5-[4-[5-Ethyl-2-Pyridyl)Ethoxy]Benzyl]-2,4-Thaizolidinedione (Formula II) by Using Cobalt (II) Nitrite

5-[4-[2-Ethyl-2-pyridyl)ethoxy]benzylidene]-2,4-thaizolidinedione (20 g), methanol (40 mL), water (140 mL) and 4% sodium hydroxide solution (26 mL) are charged into a round bottom flask and stirred for about 10 minutes. Cobalt (II) nitrite (0.25 g) and dimethyl glyoxime are dissolved in dimethyl formamide (30 mL) and added slowly for about one hour at about 20° C. to about 35° C. Mixture of sodium borohydride (3.7 g dissolved in water 40 mL), and 4% sodium hydroxide solution (9 mL) is added slowly about 3 hours at about 20° C. and stirred for about 3 hours.

Charcoal (1 g) is added into the reaction mass and stirred for about 30 minutes at about 20° C. The reaction solution is passed through high flow bed. The resultant filtrate is charged into the fresh round bottom flask and the pH of the reaction solution is adjusted to about 6.5 to about 7.0 using acetic acid (8.3 mL) and it is stirred for solid separation at about 25° C. The separated solid is filtered and washed with water (20 mL). The obtained solid is further washed with methanol (2×25 mL) and suck dried. The obtained solid is further dried at about 70° C. for about 8 hours

The obtained solid is again charged into a round bottom flask containing dimethyl formamide (80 mL) and heated to about 90° C. to get clear solution. The resultant clear solution is cooled to about 25° C. and stirred for about 60 minutes for solid separation. The separated solid is filtered and washed with dimethyl formamide (20 mL). The obtained cake is further washed with acetone and suck dried. The obtained solid is further dried at about 70° C. for about 4 hours to afford 12.7 g of the title pure compound.

### Example 6

### Purification of 5-[4-[2-Ethyl-2-Pyridyl)Ethoxy]Benzyl]-2,4-Thaizolidinedione by Using DMF and 2% Water

5-[4-[2-Ethyl-2-pyridyl)ethoxy]benzyl]-2,4-thaizolidinedione (20 g), DMF (78.4 mL) and water (1.6 mL) are charged into a round bottom flask and stirred for about 10 minutes. The resultant reaction solution is heated to about 90° C. and stirred for about 10 minutes for clear solution. The obtained clear solution is cooled to about 25° C. and stirred for solid separation. The obtained solid is filter and washed the solid with DMF (10 mL) and acetone (10 mL) and suck dried for 15 min. The solid is dried under vacuum at about 70° C. for about 5 hours to afford 18.2 g of the title pure compound.

### Example 7

### Purification of 5-[4-[2-Ethyl-2-Pyridyl)Ethoxy]Benzyl]-2,4-Thaizolidinedione by Using DMF and 4% Water

5-[4-[2-Ethyl-2-pyridyl)ethoxy]benzyl]-2,4-thaizolidinedione (20 g), DMF (76 mL) and water (4 mL) are charged into a round bottom flask and stirred for about 10 minutes. The resultant reaction solution is heated to about 90° C. and stirred for about 10 minutes for clear solution. The obtained clear solution is cooled to about 25° C. and stirred for solid separation. The solid is filter and washed the solid with DMF (10 mL) and acetone (10 mL) and suck dried for 15 min. The obtained solid is dried under vacuum at about 70° C. for about 5 hours to afford 17.9 g of the title pure compound.

### Example 8

### Purification of 5-[4-[2-Ethyl-2-Pyridyl)Ethoxy]Benzyl]-2,4-Thaizolidinedione by Using DMF and 5% Methanol

5-[4-[2-Ethyl-2-pyridyl)ethoxy]benzyl]-2,4-thaizolidinedione (20 g), DMF (76 mL) and methanol (4 mL) are charged into a round bottom flask and stirred for about 10 minutes. The resultant reaction solution is heated to about 90° C. and stirred for about 10 minutes for clear solution. The obtained clear solution is cooled to about 25° C. and stirred for solid separation. The solid is filter and washed the solid with DMF (10 mL) and acetone (10 mL) and suck dried for 15 min. The obtained solid is dried under vacuum at about 70° C. for about 5 hours to afford 18 g of the title pure compound.

### Example 9

### Purification of 5-[4-[2-Ethyl-2-Pyridyl)Ethoxy]Benzyl]-2,4-Thaizolidinedione by Using DMF and 10% Methanol

5-[4-[2-Ethyl-2-pyridyl)ethoxy]benzyl]-2,4-thaizolidinedione (20 g), DMF (72 mL) and methanol (8 mL) are charged into a round bottom flask and stirred for about 10 minutes. The resultant reaction solution is heated to about 90° C. and stirred for about 10 minutes for clear solution. The obtained clear solution is cooled to about 25° C. and stirred for solid separation. The solid is filter and washed the solid with DMF (10 mL) and acetone (10 mL) and suck dried for 15 min. The obtained solid is dried under vacuum at about 70° C. for about 5 hours to afford 18.2 g of the title pure compound.

### Example 10

### Purification of 5-[4-[2-Ethyl-2-Pyridyl)Ethoxy]Benzyl]-2,4-Thaizolidinedione by Using 1,4 DI-Oxane

5-[4-[2-Ethyl-2-pyridyl)ethoxy]benzyl]-2,4-thaizolidinedione (20 g), and 1,4-dioxane (300 mL) are charged into a round bottom flask and stirred for about 10 minutes. The resultant reaction solution is heated to about 90° C. and stirred for about 10 minutes for clear solution. The obtained clear solution is cooled to about 25° C. and stirred for solid separation. The solid is filter and washed the solid with 1,4-dioxane (20 mL) and methanol (20 mL) and suck dried for 15 min. The obtained solid is dried under vacuum at about 65° C. for about 2 hours to afford 18 g of the title compound.

HPLC Purity: 99.43%

### Example 11

### Purification of 5-[4-[2-Ethyl-2-Pyridyl)Ethoxy]Benzyl]-2,4-Thaizolidinedione by Using DMF and Isopropyl Alcohol

5-[4-[2-Ethyl-2-pyridyl)ethoxy]benzyl]-2,4-thaizolidinedione (20 g), DMF (60 mL) and IPA (60 mL) are charged into a round bottom flask and stirred for about 10 minutes. The resultant reaction solution is heated to about 90° C. and stirred for about 10 minutes. The obtained clear solution is cooled to about 25° C. and stirred for solid separation. The solid is filter and washed the solid with IPA (10 mL) and suck dried for 15 min. The obtained solid is dried under vacuum at about 70° C. for about 5 hours to afford 18 g of the title compound.

HPLC Purity: 98.98%

### Example 12

### Preparation of 5-[4-[2-Ethyl-2-Pyridyl)Ethoxy]Benzyl]-2,4-Thaizolidinedione Hydrochloride by Using Methanol (Formula I)

5-[4-[2-Ethyl-2-pyridyl)ethoxy]benzyl]-2,4-thaizolidinedione (50 g) and methanol (250 mL) are charged into a round bottom flask and stirred for about 10 minutes. Hydrochloric acid (25 mL) is added slowly into the reaction solution for about 10 minutes at about 25° C. The resultant reaction solution is heated to about 50° C. for clear solution. Charcoal (2.5 g) is added into the reaction solution and stirred for about 15 minutes at about 50° C. The obtained reaction solution is passed through high flow bed and the bed is washed with methanol (50 mL). The obtained filtrate is distilled for about 80 to about 90% below 50° C. under vacuum. To the obtained solid, acetone (125 mL) is added and it is cooled to about 0° C. The separated solid is filtered, washed with acetone (50 mL), and suck dried.

The obtained solid is again charged into the clean round bottom flask, methanol (100 mL) is charged and it is heated to about 60° C. to get clear solution. To the obtained solution, acetone (75 mL) is added and is cooled to about 0° C. for solid separation. The separated solid is filtered and washed with acetone (50 mL). The solid is dried under vacuum at about 60° C. to afford 37 g of the title compound.

HPLC purity: 99.93%

### Example 13

### Preparation of 5-[4-[2-Ethyl-2-Pyridyl)Ethoxy]Benzyl]-2,4-Thaizolidinedione Hydrochloride by Using IPA HCl (Formula I)

5-[4-[2-Ethyl-2-pyridyl)ethoxy]benzyl]-2,4-thaizolidinedione (5 g) and Isopropyl alcohol (25 mL) are charged into a round bottom flask and stirred for about 10 minutes. Hydrochloric acid (7.5 mL) is added slowly into the reaction solution for about 10 minutes at about 25° C. The resultant reaction solution is heated to about 85° C. for clear solution and stirred for about 45 min. The obtained clear solution is cooled to about 25° C. and stirred for about 45 min for solid separation. The solid is filtered and washed the solid with IPA (5 mL) and dried under vacuum to about 70° C. for 4 hours to afford 4.8 g of the title compound.

HPLC purity: 99.76%

### Example 14

### Preparation of 5-[4-[2-Ethyl-2-Pyridyl)Ethoxy]Benzyl]-2,4-Thaizolidinedione Hydrochloride by Using Water (Formula I)

5-[4-[2-Ethyl-2-pyridyl)ethoxy]benzyl]-2,4-thaizolidinedione (5 g) and water (25 mL) are charged into a round bottom flask and stirred for about 10 minutes. Hydrochloric acid (7.5 mL) is added slowly into the reaction solution for about 10 minutes at about 25° C. The resultant reaction solution is heated to about 85° C. for clear solution and stirred for about 45 min. The obtained clear solution is cooled to about 25° C. and stirred for about 45 min for solid separation. The solid is filtered and washed the solid with water (5 mL) and dried under vacuum to about 70° C. for 4 hours to afford 4.8 g of the title compound.

HPLC Purity: 99.9%

What is claimed is:

1. A process for preparing pioglitazone hydrochloride comprising the steps of:
a) reacting 5-ethyl-2-methyl pyridine with formaldehyde to afford 2-(5-ethyl-2-pyridyl)ethanol;
b) reacting 2-(5-ethyl-2-pyridyl)ethanol with methane sulfonyl chloride to afford (5-ethyl-2-pyridyl)-ethyl methanesulfonate;
c) reacting (5-ethyl-2-pyridyl)-ethyl methanesulfonate with 4-hydroxybenzaldehyde to afford 4-[2-(5-ethyl-pyridin-2-yl)-ethoxy]-benzaldehyde;
d) reacting 4-[2-(5-ethyl-pyridin-2-yl)-ethoxy]-benzaldehyde with 2,4-thiozolidinedione to afford 5-[4-[2-(5-ethyl-2-pyridyl)ethoxy]benzilidene]-2,4-thiazolidinedione; and
e) reducing 5-[4-[2-(5-ethyl-2-pyridyl)ethoxy]benzilidene]-2,4-thiazolidinedione with sodium borohydride in presence of a cobalt ion and dimethyl glyoxime to afford pioglitazone free base.

2. The process of claim 1, wherein the formaldehyde of step (a) is aqueous formaldehyde.

3. The process of claim 1, wherein the 2-(5-ethyl-2-pyridyl)ethanol and methane sulfonyl chloride of step (b) are reacted in the presence of a suitable base.

4. The process of claim 1, wherein the (5-ethyl-2-pyridyl)-ethyl methanesulfonate and 4-hydroxybenzaldehyde of step (c) are reacted in the presence of a suitable base.

5. The process of claim 1, wherein the cobalt ion of step (e) is cobalt chloride hexahydrate, cobalt (II) nitrate hexahydrate, or a combination thereof.

6. The process of claim 1, further comprising the step of recrystallizing the pioglitazone free base by using a solvent.

7. The process of claim 1, wherein the 5-[4-[2-(5-ethyl-2-pyridyl)ethoxy]benzilidene]-2,4-thiazolidinedione is reduced with sodium borohydride in the presence of a cobalt ion and dimethyl glyoxime, and in the presence of mixture of DMF and protic solvent.

8. The process of claim 7, where in the protic solvent is water, one or more alcoholic solvents, or a mixture thereof.

9. The process of claim 7, where in the amount of the protic solvent in DMF is from about 1% to about 5% by volume.

10. The process of claim 1, further comprising the step of reacting pioglitazone free base with hydrochloric acid to afford pioglitazone hydrochloride, which is free from process related impurities;
a) 5-{4-[2-(5-Ethyl-pyridin-2-yl)ethoxy]-benzylidene}-3-[2-(5-ethyl-pyridin-2-yl)-ethyl]-thiazolidine-2,4-dione of formula (XI) at relative retention time of about 1.4 RRT and;
b) 5-{4-[2-(5-Ethyl-pyridin-2-yl)-ethoxy]-benzyl}-3-[2-(5-ethyl-pyridin-2-yl)-ethyl]-thiazolidine-dione of (formula XII).
